SEQUENCE LISTING (2 sequences)

SEQ ID NO: 1 - DNA, 29 bp, Artificial Sequence
  HIV GAG Forward FAM
ataatccacctaatcccagtaggagaaat

SEQ ID NO: 2 - DNA, 27 bp, Artificial Sequence
  GAG Reverse Biotin
tttggtccttgtcttatgtccagaatg

(19) United States
(12) Patent Application Publication
(10) Pub. No.: US 2004/0038194 A1
(43) Pub. Date: Feb. 26, 2004

(54) Diagnostic polymerase chain reaction process utilizing simultaneous capture and detection of amplicons

(76) Inventor: Scott Daniel Hofmann, Plantation, FL (US)

Correspondence Address: LERNER AND GREENBERG, P.A., POST OFFICE BOX 2480, HOLLYWOOD, FL, 33022-2480, US

(21) Appl. No.: 09/974,648

(22) Filed: Oct. 9, 2001

Related U.S. Application Data
(60) Provisional application No. 60/238,792, filed on Oct. 6, 2000.

(51) Int. Cl. C12Q 1/68

(52) U.S. Cl. 435/4; 435/6

(57) ABSTRACT

A method simultaneously detects and captures double-stranded DNA sequence. The method includes providing a sample. The next step is adding a forward primer for the double-stranded DNA sequence and a reverse primer for the double-stranded DNA sequence; either the forward primer or the reverse primer have a capture agent, the other has a detection agent. The next step is replicating the double-stranded DNA sequence. The next step is binding the capture agent to a capture medium. The next step is rinsing the sample. The next step is detecting the detection agent. The method also can be applied to double-stranded DNA that has been reverse-trasncripted from single-stranded RNA.

### CROSS REFERENCE TO RELATED APPLICATIONS

This application claims the benefit of U.S. Provisional Application No. 60/238,792, filed Oct. 6, 2000.

### BACKGROUND OF THE INVENTION

1. Field of the Invention

The invention relates to qualitatively and quantitatively detecting the presence of genomic material.

The replication of genomic material (RNA and DNA) is well known and is discussed in numerous patents such as U.S. Pat. Nos. 4,582,789 and 4,683,202.

### SUMMARY OF THE INVENTION

It is an object of the invention to provide a diagnostic polymerase chain reaction utilizing simultaneous capture and detection of amplicons that overcomes the disadvantages of the heretofore-known devices and methods of this general type. With the foregoing and other objects in view there is provided, in accordance with the invention, a method for simultaneously detecting and capturing a double-stranded DNA sequence. The method includes the following steps. The first step is providing a sample. The next step is adding a forward primer for the double-stranded DNA sequence and a reverse primer for the double-stranded DNA sequence. Either the forward primer or the reverse primer have a capture agent; the other has a detection agent. The next step is replicating the double-stranded DNA sequence. The next step is binding the capture agent to a capture medium. The next step is rinsing the sample. The next step is detecting the detection agent.

In accordance with a further mode of the invention, the method includes selecting the capture agent from the group consisting of sulfhydryl group, biotin, cellulose binding domain, and a specific nucleotide sequence.

In accordance with a further mode of the invention, the capture agent includes a molecular spacer to prevent the capture agent from affecting the attached primer. In accordance with a further mode of the invention, the method includes selecting the capture medium from the group consisting of maleamide, avodin, strepavodin, cellulose, and a complementary nucleotide sequence.

In accordance with a further mode of the invention, the method includes selecting a detecting agent from the group consisting of radioactive labels, peptide antigens, and fluorometric dyes. The radioactive label can be Iodine-151.

In accordance with a further mode of the invention, the method includes adding monocolonal antibody specific to the peptide antigen having a detector.

In accordance with a further mode of the invention, the method includes selecting the detector from the group of radioactive labels, direct fluorescent antibodies, radiolabeled antibodies, and fluorometric dyes.

In accordance with a further object of the invention, the detecting agent can include a molecular spacer to prevent the capture agent from affecting the attached primer.

In accordance with a further object of the invention, the method includes detecting a plurality of double-stranded DNA sequence by adding a forward primer and a reverse primer for each additional double-stranded DNA sequence. One of each pair of a forward primers and a reverse primer has a capture agent, and the other of the pair has a second detection agent.

In addition, each detection agent is different.

In accordance with a further object of the invention, the method includes using a radioactive detection agent and detecting the detection agent with a radiodetector.

In accordance with a further object of the invention, the method includes using a fluorescence detection agent and detecting the detection agent with the fluorometer.

In accordance with a further object of the invention, the method includes detecting qualitatively the presence of the double-stranded DNA.

In accordance with a further object of the invention, the method includes detecting quantitatively the amount of the double-stranded DNA.

In accordance with a further object of the invention, the method includes replicating the double-stranded DNA using PCR.

In accordance with a further object of the invention, the method includes binding the capture agent to a stationary phase.

In accordance with a further object of the invention, the method includes binding the capture agent to a mobile phase. With the objects of the invention in view, there is also provided a method for simultaneously detecting and capturing a double-stranded DNA sequence complementing a single-stranded RNA sequence. The method includes the following steps. The first step is providing a a single-stranded RNA sequence. The next step is adding a forward primer complementing the single-stranded RNA. The next step is reverse transcripting the single-stranded RNA to produce a double-stranded DNA sequence. The next step is adding a reverse primer for the double-stranded DNA sequence; the forward primer or the reverse primer have a capture agent and the other has a detection agent. The next step is replicating the double-stranded DNA sequence. The next step is binding the capture agent to a capture medium. The next step is rinsing the sample. The next step is detecting the detection agent.

Other features that are considered as characteristic for the invention are set forth in the appended claims.

Although the invention is illustrated and described herein as embodied in a diagnostic polymerase chain reaction utilizing simultaneous capture and detection of amplicons, it is, nevertheless, not intended to be limited to the details shown since various modifications and structural changes may be made therein without departing from the spirit of the invention and within the scope and range of equivalents of the claims.

The construction and method of operation of the invention, however, together with additional objects and advantages thereof, will be best understood from the following description of specific embodiments.

### DETAILED DESCRIPTION

The invention is based on all of the normal parameters of a standard PCR or RT-PCR reaction except that the primers are modified for simultaneous capture and detection of the amplified genomic product. The reaction of PCR is based on the replication of genomic material, RNA or DNA, by the use of DNA replicating enzymes [DNA Polymerase+/−Reverse Transcriptase], suitable buffers, Nucleotide Triphosphates [i.e. DATP, dUTP, dGTP, dCTP, dTTP] and Primers.

The process requires the heating of double stranded DNA [dsDNA] to yield the two complementary strands. At this point, the forward and reverse primers attach to their complementary sites on the single stranded DNA upon cooling and the Polymerase attaches to the primer and synthesizes new dsDNA in a 5′ to 3′ direction.

My invention specifically labels one primer with a capture agent such as biotin, digoxin, cellulose binding domain, etc. and the other primer will have a detection agent such as a fluormetric dye, enzyme and substrate, radioisotope, etc. The end result of this PCR reaction will be a segment of dsDNA, from primer 1 to primer 2 [essentially forward and reverse] with one specific end designed to be captured onto a medium and the opposite end designed for detection. The method is intrinsic and simultaneous and lends itself to ease of purification after capture and prior to detection and subsequent quantification. This procedure could easily lend itself to the detection and quantification of HIV in a patient or as a low cost method for screening the blood supply. Other uses would be in detection and quantification of cancer genes in the form of their messenger RNA. Other uses for diagnostics would be multiplex screening of pathogenic viruses as well as a low cost viral load for Hepatitis B and C.

The following is an example of how this procedure may be used to detect an HIV viral load. Intravenously drawn blood is collected and 1 milliliter is diluted into a solution of RNA preservative solution. The RNA is purified out of the solution by the usual biochemical protocols with the final step eluting from a silica spin column. The RNA is eluted with RNA free water, 40 micro liters final volume, and placed into a PCR reaction tube. To this solution will be added 20 units AMV Reverse Transcriptase, 10× buffer [suitable for use with the particular PCR enzymes], and 3 units of Hot Start DNA Polymerase, 25 micromoles each of dUTP, DATP, dCTP, & dGTP. The primers are labeled as follows and are constructed in a 5′ to 3′ direction suitable for RT-PCR of HIV. HIV GAG Forward FAM [5′]-ATAATCCACCTAATCCCAGTAGGAGAAAT-[3′], mp=78C, and GAG Reverse BIOTIN [5′]-TTTGGTCCTTGTCTTATGTCCAGAATG with a mp=76C and were added to the reaction at a level of one micro molar. All of the reagents that are added to the RNA can be stored as a concentrate solution so as to impart an automated ability to the assay. An arbitrary 10 micro liters of reagent could be added to the 40 micro liters of RNA to make a 50 micro liter RT-PCR reaction. The reaction conditions are as follows: the Reverse Transcription step is allowed to occur over the course of one hour with a starting temperature of 45C and reaching a final temperature of 65C at an increase of 1 C per 3 minute, the “Hot Start” is allowed to proceed at 95 C for 15 minutes to liberate the DNA Polymerase activity, and then the following temperatures will be maintained for 40 cycles [1] 94 C for 30 seconds, [2] 55 C for 30 seconds, & [3] 72 C for 30 seconds. After the PCR, the reactants are pipetted into a strepavidin-coated plate suitable for use with a 96 well fluorometer. The binding reaction of Biotin to strepavidin is allowed to occur for a minimum of 30 minutes. The well is rinsed 3 times with Tween Tris Buffered Saline for three times [this step can be done manually or automatically]. The plate is then read for the amount of fluorescence and this should be directly proportional to the amount of virus isolated. In this particular reaction, FAM will be a 5′ labeled Flourescein for the detection of fluorescence and the Biotin, 18-space linker, will be used for capturing the Amplicons onto a strepavidin coated plate. It does not matter whether the forward or reverse primer gets the capture or detection agent as the Amplicons will still have the ability to be read and their construction is not critical. The ability of the Amplicons to be simultaneously captured, washed, and subsequently detected is what is novel about the modifications of this reaction and analysis mechanism. Although this procedure does NOT lend itself to a real-time assay, it does NOT require the expensive apparatus either. The design of this assay lends itself to automation for high throughput screening at a relatively low cost with reagents readily available.

I claim:

1. A method for simultaneously detecting and capturing a double-stranded DNA sequence, which comprises:
providing a sample;
adding a forward primer for the double-stranded DNA sequence and a reverse primer for the double-stranded DNA sequence; one of the forward primer and the reverse primer having a capture agent, the other of the forward primer and the reverse primer having a detection agent;
replicating the double-stranded DNA sequence;
binding the capture agent to a capture medium; rinsing the sample; and
detecting the detection agent.

2. The method according to claim 1, which further comprises selecting the capture agent from the group consisting of sulfhydryl group, biotin, cellulose binding domain, and a specific nucleotide sequence.

3. The method according to claim 1, wherein said capture agent includes a molecular spacer to prevent the capture agent from affecting the attached primer.

4. The method according to claim 1, which further comprises selecting the capture medium from the group consisting of maleamide, avodin, strepavodin, cellulose, and a complementary nucleotide sequence.

5. The method according to claim 1, which further includes selecting a detecting agent from the group consisting of radioactive labels, peptide antigens, and fluorometric dyes.

6. The method according to claim 5, wherein the radioactive label is Iodine-151.

7. The method according to claim 6, which further comprises adding monocolonal antibody specific to the peptide antigen having a detector.

8. The method according to claim 7, which further comprises selecting the detector from the group of radioactive labels, direct fluorescent antibodies, radiolabeled antibodies, and fluorometric dyes.

9. The method according to claim 1, wherein the detecting agent includes a molecular spacer to prevent the capture agent from affecting the attached primer.

10. The method according to claim 1, which further comprises:
detecting a plurality of double-stranded DNA sequences by:
adding a forward primer and a reverse primer for each additional double-stranded DNA sequence; one of each pair of a forward primer and a reverse primer having a capture agent, and the other of the pair having a second detection agent, each detection agent being different.

11. The method according to claim 1, which further comprises using a radioactive detection agent and detecting the detection agent with a radiodetector.

12. The method according to claim 1, which further comprises using a fluorescence detection agent and detecting the detection agent with the fluorometer.

13. The method according to claim 1, which further comprises detecting qualitatively the presence of the double-stranded DNA.

14. The method according to claim 1, which further comprises detecting quantitatively the amount of the double-stranded DNA.

15. The method according to claim 1, wherein the replicating step comprises replicating the double-stranded DNA using PCR.

16. The method according to claim 1, which further comprises binding the capture agent to a stationary phase.

17. The method according to claim 1, which further comprises binding the capture agent to a mobile phase.

18. A method for simultaneously detecting and capturing a double-stranded DNA sequence complementing a single-stranded RNA sequence, which comprises:
providing a a single-stranded RNA sequence;
adding a forward primer complementing the single-stranded RNA;
reverse transcripting the single-stranded RNA to produce a double-stranded DNA sequence;
adding a reverse primer for the double-stranded DNA sequence;
one of the forward primer and the reverse primer having a capture agent, the other of the forward primer and the reverse primer having a detection agent;
replicating the double-stranded DNA sequence;
binding the capture agent to a capture medium;
rinsing the sample; and
detecting the detection agent.
